(19) United States
(12) Patent Application Publication
(10) Pub. No.: US 2025/0230110 A1
(43) Pub. Date: Jul. 17, 2025

(54) A PROCESS FOR BUTANE HYDROGENOLYSIS HAVING SAFE THERMAL OPERATION

(71) Applicant: SABIC Global Technologies B.V., Bergen op Zoom (NL)

(72) Inventors: Byeongjin BAEK, Bangalore (IN); Dustin FICKEL, Bangalore (IN); Emiel VAN KIMMENADE, Bangalore (IN); Tian GU, Bangalore (IN)

(73) Assignee: SABIC Global Technologies B.V., Bergen op Zoom (NL)

(21) Appl. No.: 18/855,895

(22) PCT Filed: Apr. 24, 2023
(86) PCT No.: PCT/EP2023/060596

§ 371 Date: Oct. 10, 2024

(30) Foreign Application Priority Data
May 2, 2022 (EP) 22171195.5
Nov. 9, 2022 (EP) 22206481.8

(51) Int. Cl. C07C 4/06; C07C 7/00

(52) U.S. Cl. CPC C07C 4/06; C07C 7/00; C07C 2521/04; C07C 2523/46; C07C 2529/06

(57) ABSTRACT

The invention relates to a process for butane hydrogenolysis for producing one or more desired hydrogenolysis products. The process involves introducing an initial feed stream comprising a butane feed and hydrogen feed in a reactor system comprising a number of reactors, for example not more than 10 reactors, arranged in a series. Each of these reactors contain a butane hydrogenolysis catalyst loaded at specific catalyst loading in a manner such that every reactor apart from the first reactor in the series has higher catalyst loading than the catalyst loading of the immediately preceding reactor. Further, each of the reactors are configured to operate at a higher reactor temperature rise than the preceding reactor. The process may include the use of diluent, preferably methane, with the feed stream in one or more of the reactors. The invention further relates to a reactor system process for conducting the process for butane hydrogenolysis.

### CROSS REFERENCE TO RELATED APPLICATIONS

This application claims priority to European Application No. 22171195.5 filed on May 2, 2022 and to European Application No. 22206481.8 filed on Nov. 9, 2022.

### FIELD OF INVENTION

The invention relates to the field of butane hydrogenolysis. In particular the invention is directed to a process of butane hydrogenolysis and to a reactor system suitable for carrying out butane hydrogenolysis.

### BACKGROUND

Butane is used extensively in the chemical industry as part of the process to produce natural gas, distillates, and other refinery products. For example, light alkenes and alkanes may be obtained from a butane (C4) stream through thermal steam cracking, hydrocracking reactions, and/or through hydrogenolysis reactions. However, it has been found that thermal steam cracking of a butane feedstock produces relatively low yield of olefins including ethylene and propylene. Hence, as an alternate to thermal steam cracking, catalytic butane hydrogenolysis has been proposed to increase the yield and selectivity of high value hydrocarbon products such as ethane.

For ensuring that the butane hydrogenolysis process is efficient, butane conversion butane should be high (e.g. 90% and above) while maintaining excellent ethane selectivity (e.g. 70% and above). High butane conversion level and ethane selectivity can be achieved by either increasing the temperature rise in a reactor (reactor temperature rise) or by increasing the number of reactors arranged in series. As butane hydrogenolysis involves highly exothermic reactions, the option of maximizing the reactor temperature rise may not be suitable, as beyond a point such reactor temperature rise may result in a thermal runaway.

One of the ways of adjusting the reactor temperature rise is by controlling the temperature at the inlet of a reactor when the feed is introduced. However, owing to the highly exothermic chemistry, the performance of butane hydrogenolysis is sensitive to even a small fluctuation of temperature especially at the inlet of a reactor, which in certain instances leads to thermal runway. Consequences of thermal runaway is that not only the selectivity for ethane and other desired products, is adversely affected but also such thermal runaway may raise serious safety concerns during ordinary plant operations.

Therefore, to ensure a safer thermal operation while ensuring high butane conversion and product selectivity, operational limits are placed to limit the temperature rise per reactor. As an alternate approach, butane conversion and ethane selectivity can be increased by maximizing the number of reactors in a series. However, the limit in the number of reactors is governed by capital and operational cost consideration. Accordingly, it is desired that there is a balance between maximizing the reactor temperature rise, and the number reactors that are deployed for hydrogenolysis while achieving a sufficiently high butane conversion and product selectivity without the risks of thermal runaway.

In the past several publications have been made available to pubic related to butane hydrogenolysis. US202203335 relates to a process for the hydrogenolysis of butane. The patent publication discloses that the process can include controlling the introduction of a butane feed stream and hydrogen to a first hydrogenolysis reactor such that the hydrogen to butane molar ratio in the reactor is controlled from 0.3:1 to 0.8:1.

WO2020061012 relates to methods of processing butanes, more specifically methods of reactive separation of n-butane and i-butane.

Therefore, one of the objectives of the present invention is to provide a butane hydrogenolysis process that achieves a suitable level of butane conversion with high ethane selectivity while mitigating the risks of thermal runaway. Another objective of the invention is to provide a butane hydrogenolysis process, which is configured to mitigate thermal runaway even when there is a minor fluctuation of temperature at a reactor inlet. Yet another objective of the present invention is to provide a reactor system suitable for carrying out butane hydrogenolysis involving a minimum number of fixed bed reactors arranged in series, that are configured to mitigate thermal runaway while achieving sufficiently high butane conversion and ethane selectivity. Yet another objective of the present invention is to provide a diluent that is added to the butane hydrogenolysis process to mitigate thermal runaway while achieving sufficiently high butane conversion. Yet another objective of the present invention is to provide a diluent that is added to the reactor system suitable for carrying out butane hydrogenolysis to mitigate thermal runaway while achieving sufficiently high butane conversion.

### SUMMARY

Accordingly, the one or more objectives of the invention is achieved by a process for butane hydrogenolysis for producing one or more desired hydrogenolysis products, where the process includes the steps of.
a) providing a reactor system comprising a plurality of ‘N’ reactors arranged in a series, wherein ‘N’ is an integer ≤10, preferably ≤5; wherein the first reactor of the series is denoted as R₁, the last reactor of the series is denoted as R_{N}, the penultimate reactor of the series is denoted as R_{N-1} and the remaining reactors are denoted serially from 2 to (N-2);
b) introducing a feed stream comprising a butane feed and hydrogen feed, in a ‘X’ reactor denoted as R_{X}, and producing a (X)^{th} hydrogenolysis product stream under conditions of butane hydrogenolysis; wherein the (X)^{th} hydrogenolysis product stream comprises an unreacted butane feed, hydrogenolysis products, and unreacted hydrogen;
   i. wherein R_{X} is any one of the reactors arranged in the series from reactor R₁ to reactor R_{N-1};
   ii. wherein ‘X’ is an integer ranging from ≥1 and ≤N-1, wherein when ‘X’=1, the feed stream introduced in the first reactor R₁ is an initial feed stream being introduced into the reactor system;
c) introducing at least a portion of the (X)^{th} hydrogenolysis product stream to an immediate subsequent reactor, a (X+1) reactor denoted as R_{X+1}, and producing a (X+1)^{th} hydrogenolysis product stream under conditions of butane hydrogenolysis; wherein the (X+1)^{th} hydrogenolysis product stream comprises an unreacted butane feed, hydrogenolysis products, and unreacted hydrogen;
   i. wherein the reactor R_{X+1} is an immediately subsequent reactor to the reactor R_{X};
d) provided if ‘N’ is >2, repeating the process steps of b) and c) for a further N-2 times until a N^{th} hydrogenolysis product stream is produced in the R_{N} reactor; wherein the N^{th} hydrogenolysis product stream comprises unreacted butane, hydrogenolysis products, and unreacted hydrogen, provided that a feed stream introduced in the reactor R_{X+1} comprises at least a portion of the (X)^{th} hydrogenolysis product stream that is produced in the immediately preceding reactor R_{X},
   i. provided if ‘N’ is 2, a second hydrogenolysis product stream produced by a second reactor R₂ is the N^{th} hydrogenolysis product stream;
e) recovering at least a portion of the hydrogenolysis products from the N^{th} hydrogenolysis product stream and obtaining one or more desired hydrogenolysis products;
   wherein each of the reactors in series from R₁ to R_{N} comprises:
      i. a reactor vessel; and
      ii. a catalyst bed disposed in the reactor vessel, wherein the catalyst bed comprises: a butane hydrogenolysis catalyst, wherein the butane hydrogenolysis catalyst is loaded at a catalyst loading (CL); wherein each reactor from R₂ to R_{N} has a catalyst loading (CL) that is ≥5% and ≤150%, preferably ≥15% and ≤120%, preferably ≥25% and ≤110%, preferably ≥30% and ≤80%, higher than the catalyst loading of the immediately preceding reactor;
      iii. wherein the process is performed such that each reactor from R₂ to R_{N} has a reactor temperature rise of ≥1° C. and ≤50° C., preferably ≥2° C. and ≤40° C., preferably ≥5° C. and ≤35° C. higher than the reactor temperature rise of the immediately preceding reactor; wherein the one or more desired hydrogenolysis products are selected from ethane, propane, methane and mixtures thereof.

In some embodiments of the invention, each reactor from R₂ to R_{N} is operated at a butane feed stream based weight hourly space velocity (WHSV) of ≥5% and ≤70%, preferably ≥10% and ≤60%, preferably ≥20% and ≤60%, lower than the butane feed stream based weight hourly space velocity (WHSV) being operated at the immediately preceding reactor; and/or the process further comprises introducing a diluent into one or more of the R₁ to R_{N} reactors, preferably wherein the diluent is methane.

Preferably, the process further comprises the step of introducing a diluent into one or more of the R₁ to R_{N} reactors. Preferably, diluent is more than 80% preferably more than 90%, preferably more than 100%, preferably more than 120%, preferably more than 140%, with regard to butane concentration present in the reactor in which the diluent is introduced.

In some embodiments of the invention, when X=1, the second reactor R₂ has ≥25% and ≤110%, preferably ≥35% and ≤80%, preferably ≥40% and ≤70%, preferably ≥50% and ≤70%, of higher catalyst loading than the catalyst loading of the first reactor R₁.

In some embodiments of the invention, the step of recovering at least a portion of the hydrogenolysis products from the N^{th} hydrogenolysis product stream, comprises:
a) feeding at least a portion of the N^{th} hydrogenolysis product stream to a separation unit and forming a hydrogenolysis product stream, an unreacted hydrogen stream, a separated butane stream comprising i-butane and n-butane;
b) recovering at least a portion of the hydrogenolysis products from the hydrogenolysis product stream and obtaining one or more desired hydrogenolysis products;
c) feeding the separated butane stream into an isomerization unit and obtaining a n-butane rich stream; and
d) recirculating back at least a portion of the n-butane rich stream and the unreacted hydrogen stream to one or more reactors R₁ to R_{N}.

In some embodiments of the invention, the first reactor R₁ is operated under a condition of reactor temperature rise of ≤110° C., preferably ≤100° C., preferably ≤90° C., preferably ≤70° C., preferably ≤60° C., preferably ≤50° C., preferably ≤40° C., when an initial reactor inlet temperature (Tᵢₙₗₑₜ₁) of the first reactor R₁ is increased by an amount of ≥0.5° C. and ≤3.0° C., preferably ≥1.0° C. and ≤3.0° C., preferably ≥2.0° C. and ≤2.5° C., wherein the reactor temperature rise is determined as (Tₒᵤₜₗₑₜ₁-Tᵢₙₗₑₜ₁) where Tₒᵤₜₗₑₜ is the outlet reactor temperature of the first reactor R₁ and Tᵢₙₗₑₜ₁ is the initial inlet temperature of the first reactor R₁.

In some embodiments of the invention, the one or more desired hydrogenolysis products are selected from ethane, propane, methane and mixtures thereof, preferably the one or more desired hydrogenolysis product is ethane produced at a selectivity of ≥60.0%, preferably ≥65.0%, preferably ≥70.0%, preferably ≥75.0%, with regard to a total molar concentration of all hydrogenolysis products.

In some embodiments of the invention, an additional butane feed and/or hydrogen feed is introduced into the (X+1) reactor (R_{X+1}), along with the (X)^{th} hydrogenolysis product stream. In some embodiments of the invention, the butane feed and/or the unreacted butane feed comprises a mixture of n-butane and/or i-butane.

In some embodiments of the invention, feed stream comprising a butane feed and hydrogen feed may further comprise minor amounts of hydrocarbons comprising hydrocarbons having three, or five or six or eight carbon atoms.

In some embodiments of the invention, the ratio of the molar concentration of n-butane present in the N^{th} hydrogenolysis product stream to the molar concentration of n-butane present in the initial feed stream is ≤0.4, preferably ≤0.3, preferably ≤0.2, preferably ≤0.1, preferably ≤0.05. In some embodiments of the invention, the ratio of the molar concentration of n-butane present in a first hydrogenolysis product stream produced in the first reactor R₁ (first hydrogenolysis product stream) to the molar concentration of n-butane present in the initial feed stream is ≤0.95, preferably ≤0.85, preferably ≤0.70, preferably ≤0.5.

In some embodiments of the invention, the step of recovering at least a portion of the hydrogenolysis products from the N^{th} hydrogenolysis product stream, comprises the step of feeding at least a portion of the N^{th} hydrogenolysis product stream to a hydrocracking reactor and obtaining a hydrocracking product stream comprising one or more desired hydrogenolysis products.

In some embodiments of the invention, the butane hydrogenolysis catalyst is selected from:
(i) a bimetallic supported catalyst comprising a support, a first catalytic metal, a second catalytic metal, and optionally a binder, wherein the first catalytic metal and the second catalytic metal are different,
(ii) a monometallic supported catalyst, the monometallic catalyst comprising a third catalytic metal, a support, and optionally binder, or
(iii) mixtures of (i) and (ii), wherein the first catalytic metal, the second catalytic metal, and the third catalytic metal are each independently selected from iridium (Ir), platinum (Pt), rhodium (Rh), ruthenium (Ru), rhenium (Re), palladium (Pd), molybdenum (Mo), tungsten (W), nickel (Ni), or cobalt (Co), or any combination thereof.

In some embodiments of the invention, the butane hydrogenolysis catalyst comprises the bimetallic supported catalyst comprising Ir and Pt and wherein the support comprises alumina, a zeolite, or both, wherein the zeolite comprises ZSM-5, ZSM-11, Y, high-silica Y, USY, EU-1, EU-2, beta, L, ferrierite, CHA, SSZ-16, Nu-3, sigma-1, or silicalite-1, or any combination thereof, and optionally wherein the binder if present, is selected from alumina, titania, silica, or combinations thereof.

In some embodiments of the invention, the reactor system comprises a plurality of 4 reactors (N=4) arranged in a series, wherein the first reactor of the series is denoted as R₁, the fourth reactor of the series is denoted as R₄, and the remaining reactors in the series are denoted as R₂ and R₃, wherein the second reactor R₂ has ≥25% and ≤110%, preferably ≥35% and ≤80%, preferably ≥40% and ≤70%, preferably ≥50% and ≤70%, of higher catalyst loading than the catalyst loading of the first reactor R₁, wherein the initial feed stream is introduced into the first reactor (R₁) and one or more desired hydrogenolysis products is obtained from a fourth hydrogenolysis product stream produced in the fourth reactor R₄.

In some preferred embodiments of the invention, the invention is directed to a reactor system for conducting the process for butane hydrogenolysis in accordance with one or more embodiments of the present invention, wherein the reactor system comprises a plurality of ‘N’ reactors arranged in a series, wherein ‘N’ is an integer ≤10, preferably ≤5; wherein the first reactor of the series is denoted as R₁, the last reactor of the series is denoted as R_{N}, the penultimate reactor of the series is denoted as R_{N-1} and the remaining reactors are denoted serially from 2 to (N-2);
a) wherein each of the reactors ranging from R₁ to R_{N} comprises:
   i. a reactor vessel; and
   ii. a catalyst bed disposed in the reactor vessel and extending along a flow direction of reactants, wherein the catalyst bed comprises a butane hydrogenolysis catalyst loaded at a catalyst load (CL); wherein each reactor from R₂ to R_{N} has a catalyst loading (CL) that is ≥5% and ≤150%, preferably ≥15% and ≤120%, preferably ≥25% and ≤110%, preferably ≥30% and ≤80%, higher than the catalyst loading of the immediately preceding reactor;
b) the first reactor R₁ is configured to operate under a condition of reactor temperature rise of ≤110° C., preferably ≤100° C., preferably ≤90° C., when an initial reactor inlet temperature (Tᵢₙₗₑₜ₁) of the first reactor R₁ is increased by an amount of ≥0.5° C. and ≤3.0° C., preferably ≥1.0° C. and ≤3.0° C., preferably 2.0° C. and ≤2.5° C., wherein the reactor temperature rise is determined as (Tₒᵤₜₗₑₜ-Tᵢₙₗₑₜ₁) where Tₒᵤₜₗₑₜ₁ is the outlet reactor temperature of the first reactor R₁ and Tᵢₙₗₑₜ₁ is the initial inlet temperature of the first reactor R₁; wherein the first reactor R₁ is configured to receive the initial feed stream; and
c) wherein the reactor system is configured to achieve an overall n-butane conversion of ≥60.0%, preferably ≥70.0%, preferably ≥80.0%, preferably ≥95.0%; and
d) wherein each reactor from R₂ to R_{N} is configured to have a reactor temperature rise of ≥1° C. and ≤50° C., preferably ≥2° C. and ≤40° C., preferably ≥5° C. and ≤35° C., higher than the reactor temperature rise of the immediately preceding reactor; and optionally wherein at least of one of the reactors R₁ to R_{N} has an inlet to receive a diluent gas.

Preferably, at least of one of the reactors R₁ to R_{N} has an inlet to receive a diluent gas.

In an embodiment of the invention, the invention the invention is directed to a reactor system that includes a feedstream or other commonly known means to introduce a diluent, preferably methane, into the hydrogenolysis reactor. It can be a separate feedstream fed directly into the hydrogenolysis reactor or it can be a feedstream is premixed with the butane and hydrogen feed. In this way, there is either a separate inlet in the hydrogenolysis reactor for the diluent feed or an inlet in either the butane feed, the hydrogen feed, or the combined butane/hydrogen feed, depending on the configuration. The added diluent acts as a heat carrier, which reduces the temperature rise over the same butane conversion.

Methane is the preferred diluent. The addition of methane diluent provides the added benefit that with the same temperature rise limit, the dilution can increase the butane conversion per reactor safely. This can also reduce the number of reactors required to achieve the same overall butane conversion. In other embodiments of the invention, the invention the invention is directed to a process that includes introducing a diluent, preferably methane, into the hydrogenolysis process. The added methane acts as a heat carrier, which reduces the temperature rise over the same butane conversion. The addition of methane diluent provides the added benefit that with the same temperature rise limit, the dilution can increase the butane conversion per reactor safely. This can also reduce the number of reactors required to achieve the same overall butane conversion.

Other objects, features and advantages of the invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from some specific embodiments may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 is a schematic diagram for a process of butane hydrogenolysis in accordance with an embodiment of the present invention involving four fixed bed adiabatic reactors in series coupled to a separation unit and an isomerization unit for the recovery of the one or more desired hydrogenolysis product.

FIG. 2 is a schematic diagram for a process of butane hydrogenolysis in accordance with an embodiment of the present invention involving four fixed bed adiabatic reactors in series coupled to a hydrocracking unit for the recovery of the one or more desired hydrogenolysis product.

### DETAILED DESCRIPTION

The following includes definitions of various terms, expressions and phrases used throughout this specification.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.” The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The method of the invention can “comprise,” “consist essentially of,” or “consist of” particular ingredients, components, compositions, etc., disclosed throughout the specification.

In the context of the present invention, the hydrogenolysis of butane involves the reaction schemes as shown in (1)-(3):

n-C₄H₁₀+H₂→2C₂H₆ (1)

n-C₄H₁₀+H₂→C₃H₈+CH₄ (2)

i-C₄H₁₀+H₂→C₃H₈+CH₄ (3).

Reaction scheme 1 is the desired hydrogenolysis reaction, while reaction schemes 2 and 3 show the side reactions. Accordingly, the term “one or more desired hydrogenolysis products” as used throughout this disclosure means the production of ethane and optionally minor amounts of propane and methane through the process of butane hydrogenolysis. More particularly, the expression “one or more desired hydrogenolysis products” as used throughout this disclosure means hydrocarbon products selected from the group consisting of ethane, propane, methane and mixtures thereof.

The expression “catalyst loading” or “catalyst load” as used interchangeably throughout this disclosure means the total weight of the butane hydrogenolysis catalyst including the weight of the catalyst support and optionally a binder normalized to reactor volume, that is present in the reactor bed for a particular reactor and relative to the reactor volume. For the case of identical reactors in series, the catalyst loading is simply the mass of catalyst.

The term “reactor temperature rise” for a given reactor as used throughout this disclosure means the rise in temperature inside a reactor due to the exothermic hydrogenolysis reaction being carried out in the reactor. The reactor temperature rise measures the rise in the temperature inside the reactor from the time period of introducing the butane feed and hydrogen feed stream in the reactor until the time the hydrogenolysis products that is produced is removed from the reactor. The reactor temperature rise is determined as (Tₒᵤₜₗₑₜ−Tᵢₙₗₑₜ) where Tₒᵤₜₗₑₜ is the final outlet reactor temperature when the hydrogenolysis products are removed from a particular reactor and Tᵢₙₗₑₜ is the initial inlet temperature measured at the time of introducing the feed stream.

The term “subsequent reactor” means a reactor positioned adjacent to a preceding reactor, which receives a hydrogenolysis product stream produced by a preceding reactor.

The term “preceding reactor” means a reactor positioned adjacent to a preceding reactor, which produces a hydrogenolysis product stream to be introduced in the subsequent reactor.

The term “thermal runaway” means the reactor temperature rise exceeding 110° C. during the course of the hydrogenolysis reaction or the reactor temperature rise inside a reactor when there is a fluctuation of temperature at the inlet of a reactor.

The term “at least a portion” as used throughout this disclosure with regard to any feed stream or a hydrogenolysis product stream means ≥80.0%, preferably ≥95.0%, preferably ≥99.0%, preferably 100.0%, of the stream.

The term “plurality of ‘N’ reactors arranged in a series” means an arrangement of ‘N’ number of reactors arranged in series such that the initial feed stream is introduced in the first reactor in the series and every reactor in the series produces a hydrogenolysis product stream, which is introduced as part of the feed to the subsequent reactor till the final hydrogenolysis product stream is recovered from the last or the N^{th} reactor in the series. The purpose of introducing the variable ‘N’ is to define the total number of reactors which are involved in the process of butane hydrogenolysis. For the purpose of the present invention, the first reactor R₁ is the first reactor of the series where the initial feed stream is introduced in the reactor for initiating the process of butane hydrogenolysis. For the purpose of the present invention, the last reactor of the series is R_{N} where the N^{th} hydrogenolysis product stream is produced and subsequently from which the one or more hydrogenolysis products are recovered.

The term “‘X’ reactor denoted as R_{X}” is to define a particular reactor in the series, where R_{X} is any one of the reactors arranged in the series from reactor R₁ to reactor R_{N-1}. The purpose of defining the reactor R_{X} is to indicate the steps of the butane hydrogenolysis process involving a reactor, where each of the reactors falling under the definition of R_{X} produces a hydrogenolysis product stream upon receiving a feed stream.

The term “(X+1) reactor denoted as R_{X+1}” means a reactor that is subsequent to the reactor R_{X}. The reactor R_{X+1} is configured to receive the hydrogenolysis product stream. The reactor defined as R_{X+1} is defined relative to the reactor R_{X}.

The term “N^{th} hydrogenolysis product stream” means the last hydrogenolysis product stream produced by the last reactor arranged in the series involving ‘N’ reactors.

The invention is based, in part, on the discovery of a process for carrying out butane hydrogenolysis for producing one or more desired hydrogenolysis products. The process of the present invention involves the use of a number of fixed bed reactors arranged in series such that each of the subsequent reactors apart from the first reactor has a progressively increased reactor temperature rise and a progressively increased catalyst load compared to the immediate preceding reactor in the series.

For example, in accordance with the present invention, every reactor in the series apart from the first reactor, may have a ≥5% and ≤150%, preferably ≥15% and ≤120%, preferably ≥25% and ≤110%, preferably ≥30% and ≤80%, of higher catalyst loading compared to the catalyst loading of the immediately preceding reactor vessel in the series and a ≥1° C. and ≤50° C., preferably ≥2° C. and ≤40° C., preferably ≥5° C. and ≤35° C. of higher reactor temperature rise than the reactor temperature rise of the immediately preceding reactor. Progressive increase of the catalyst loading and reactor temperature rise across the series of reactors by an amount as prescribed in the present invention, reduced the risk of thermal runaway significantly while ensuring high butane conversion along with ethane selectivity.

Accordingly, in some aspects of the invention, the invention relates to a process for butane hydrogenolysis for producing one or more desired hydrogenolysis products, where the process includes the steps of:
a) providing a reactor system comprising a plurality of ‘N’ reactors arranged in a series, wherein ‘N’ is an integer ≤10, preferably ≤5; wherein the first reactor of the series is denoted as R₁, the last reactor of the series is denoted as R_{N}, the penultimate reactor of the series is denoted as R_{N-1} and the remaining reactors are denoted serially from 2 to (N-2);
b) introducing a feed stream comprising a butane feed and hydrogen feed, in a ‘X’ reactor denoted as R_{X}, and producing a (X)^{th} hydrogenolysis product stream under conditions of butane hydrogenolysis; wherein the (X)^{th} hydrogenolysis product stream comprises an unreacted butane feed, hydrogenolysis products, and unreacted hydrogen;
   i. wherein R_{X} is any one of the reactors arranged in the series from reactor R₁ to reactor R_{N-1};
   ii. wherein ‘X’ is an integer ranging from ≥1 and ≤N-1,
   iii. wherein when ‘X’=1, the feed stream introduced in the first reactor R₁ is an initial feed stream being introduced into the reactor system;
c) introducing at least a portion of the (X)^{th} hydrogenolysis product stream to an immediate subsequent reactor, a (X+1) reactor denoted as R_{X+1}, and producing a (X+1)^{th} hydrogenolysis product stream under conditions of butane hydrogenolysis; wherein the (X+1)^{th} hydrogenolysis product stream comprises an unreacted butane feed, hydrogenolysis products, and unreacted hydrogen;
   i. wherein the reactor R_{X+1} is an immediately subsequent reactor to the reactor R_{X};
d) provided if ‘N’ is ≥2, repeating the process steps of b) and c) for a further N-2 times until a N^{th} hydrogenolysis product stream is produced in the R_{N} reactor; wherein the N^{th} hydrogenolysis product stream comprises unreacted butane, hydrogenolysis products, and unreacted hydrogen, provided that a feed stream introduced in the reactor R_{X+1} comprises at least a portion of the (X)^{th} hydrogenolysis product stream that is produced in the immediately preceding reactor R_{X};
   i. provided if ‘N’ is 2, the second hydrogenolysis product stream produced by the second reactor R₂ is the N^{th} hydrogenolysis product stream;
e) recovering at least a portion of the hydrogenolysis products from the N^{th} hydrogenolysis product stream and obtaining one or more desired hydrogenolysis products. Further, each of the reactors in series from R₁ to R_{N} comprises:
   i. a reactor vessel;
   ii. a catalyst bed disposed in the reactor vessel, wherein the catalyst bed comprises: a butane hydrogenolysis catalyst, wherein the butane hydrogenolysis catalyst is loaded at a catalyst loading (CL); wherein each reactor from R₂ to R_{N} has a catalyst loading (CL) that is ≥5% and ≤150%, preferably ≥15% and ≤120%, preferably ≥25% and ≤110%, preferably ≥30% and ≤80% higher than the catalyst loading of the immediately preceding reactor; and
   iii. wherein the process is performed such that each reactor from R₂ to R_{N} has a reactor temperature rise of ≥1 C and ≤50° C., preferably ≥2° C. and ≤40° C., preferably ≥5° C. and ≤35° C. higher than the reactor temperature rise of the immediately preceding reactor; wherein the one or more desired hydrogenolysis products are selected from ethane, propane, methane and mixtures thereof.

For carrying out the process of butane hydrogenolysis a suitable rector system may be used. For example, the reactor system may comprise a plurality of ‘N’ reactors arranged in a series, wherein ‘N’ is an integer ≤10, preferably ≤5; wherein the first reactor of the series is denoted as R₁, the last reactor of the series is denoted as R_{N}, the penultimate reactor of the series is denoted as R_{N-1} and the remaining reactors are denoted serially from 2 to (N-2). Preferably, ‘N’ is an integer ranging from 1> and ≤10, preferably ‘N’ is an integer ranging from 1> and ≤5, preferably ‘N’ is an integer ranging from 1> and ≤4.

Preferably, in some embodiments of the invention, each of the ‘N’ reactors arranged in series (1 to N) is operated adiabatically. Preferably, in some embodiments of the invention, each of the reactors in series is a fixed bed adiabatic reactor. In some embodiments of the invention, each of the reactors R₁ to R_{N} have equivalent dimensions including equivalent reactor volume. In some embodiments of the invention, at least one or more reactors have a dimension including reactor volume that is different from the remaining set of reactors. In such cases, that catalyst loading would be the mass of catalyst (kg) normalized to the appropriate reactor dimension such as volume of the reactor.

In some embodiments of the invention, for example, if the number of reactors arranged in series is 10 then ‘N’ is 10. Accordingly, the first reactor is denoted as R₁, the penultimate reactor is denoted as R₉ and the last reactor of the series is denoted as Rio. The remaining reactors may be denoted from R₂ to R₈. Similarly, if the number of reactors arranged in series is 4 then ‘N’ is 4. Accordingly, the first reactor is denoted as R₁, the penultimate reactor is denoted as R₃ and the last reactor of the series is denoted as R₄. The remaining reactors may be denoted as R₂.

The initial feed stream comprising the butane feed stream and the hydrogen feed is present at a suitable proportion of the reactants. The butane feed comprises for example n-butane and i-butane. In some embodiments of the invention, the flow of butane feed (e.g., and butane WHSV) and hydrogen feed to the hydrogenolysis reactor system can be controlled (e.g., flow meters, valves, and the like) to maintain a hydrogen to butane (n-butane and i-butane) molar ratio in the first reactor R₁ below 1, preferably between 0.3:1 and 0.8:1, or 0.3:1, 0.35:1, 0.4:1, 0.45:1, 0.5:1, 0.55:1. 0.60:1, 0.65:1, 0.70:1, 0.75:1, 0.80:1, or any range or value there between. The butane feed stream can include a mixture of butanes (e.g., n-butane and i-butane) and minimal amounts of other hydrocarbons.

In some embodiments of the invention, the butane feed and/or the unreacted butane feed comprises a mixture of n-butane and/or i-butane. The initial feed stream being introduced in the first reactor R₁ has a suitable molar concentration of n-butane and i-butane. For example, the butane feed stream can include a mixture of butanes (e.g., n-butane and i-butane) and minimal amounts of other hydrocarbons.

In some embodiments of the invention, the butane feed stream may comprise minor amounts of other hydrocarbons such hydrocarbons may for example have three carbon atoms (C₃ hydrocarbons), or five carbon atoms (C₅ hydrocarbons), six carbon atoms (C₆ hydrocarbons) along with n-butane. The n-butane can be present in the butane feed stream in an amount of ≥50.0 mol %. For example, the amount of n-butane may be ≥50.0 mol. %, ≥55.0 mol. %, ≥60.0 mol. %, 65.0 mol. %, ≥70.0 mol. %, 75.0 mol. %, ≥80.0 mol. %, 85.0 mol. %, ≥90.0 mol. %, ≥95.0 mol. %, ≥99.9 mol. % or any range or value there between. In some embodiments of the invention, the amount of n-butane can be ≥50 mol. % and ≤99.0 mol. %, ≥55.0 mol. % and ≤90.0 mol. %, 60.0 mol. % and ≤85.0 mol. %, 65.0 mol. % and ≤80.0 mol. %, or ≥70.0 mol. % and ≤75.0 mol. % or any range there between.

The feed stream comprising a butane feed and hydrogen feed may for example be introduced in a ‘X’ reactor denoted as R_{X}, and assesses a (X)^{th} hydrogenolysis product stream; wherein the (X)^{th} hydrogenolysis product stream comprises an unreacted butane feed, hydrogenolysis products, and unreacted hydrogen; wherein R_{X} is any one of the reactors arranged in the series from reactor R₁ to reactor R_{N-1}; wherein ‘X’ is an integer ranging from ≥1 and ≤N-1, wherein when X=1, the feed stream introduced in the first reactor R₁ is an initial feed stream being introduced into the reactor system.

For example, when the number of reactors arranged in series is 4 then N=4. The reactor R_{X} may for example be any of the reactors R₁, R₂, and R₃. The initial feed stream comprising butane feed and hydrogen may for example be introduced in the first reactor R₁. Each of the reactors R₁, R₂ and R₃ may for example produce the corresponding hydrogenolysis product stream comprising an unreacted butane feed, hydrogenolysis products, and unreacted hydrogen. The first reactor R₁ may for example produces a first hydrogenolysis product stream, the reactor R₂ produces a second hydrogenolysis product stream and the reactor R₃ produces a third hydrogenolysis product stream.

For every (X)^{th} hydrogenolysis product stream once produced, at least a portion of the (X)^{th} hydrogenolysis product stream may be introduced to an immediate subsequent reactor, a (X+1) reactor denoted as R_{X+1}, and producing a (X+1) hydrogenolysis product stream under conditions of butane hydrogenolysis. For example, when the number of reactors arranged in series is 4 (N=4), for the first round when X=1, the first hydrogenolysis product stream ((X)^{th} hydrogenolysis product stream) produced from the first reactor R₁ (R_{X}) under conditions of butane hydrogenolysis, is introduced into the second reactor R₂ (R_{X+1}) and subsequently a second hydrogenolysis product stream ((X+1)^{th} hydrogenolysis product stream) is produced under conditions of butane hydrogenolysis.

In accordance with the present invention, if the number of reactors arranged is greater than 2 (i.e N is ≥2), process steps involving the production of (X)^{th} hydrogenolysis product stream and the production of (X+1)^{th} hydrogenolysis product stream is repeated for a further N-2 times until a N^{th} hydrogenolysis product stream is produced in the last or final reactor (R_{N} reactor). In other words, the process is repeated until the (X+1)^{th} hydrogenolysis product stream is the N^{th} hydrogenolysis product stream. Accordingly, the (N-1) hydrogenolysis product stream produced by the penultimate reactor R_{N-1} is introduced into the last or final reactor in the series R_{N} which in turn produces the N^{th} hydrogenolysis product stream.

Accordingly, if N=4, there would a further two rounds of operation. For example, for the second round or X=2, the second hydrogenolysis product stream ((X)^{th} hydrogenolysis product stream) is introduced into the third reactor R₃ (X+1 reactor) and subsequently a third hydrogenolysis product stream ((X+1)^{th} hydrogenolysis product stream) is produced under conditions of butane hydrogenolysis.

For the third round, for X=3, the third hydrogenolysis product stream ((X)^{th} hydrogenolysis product stream) produced by the third reactor R₃ (R_{X}) is introduced into the fourth reactor R₄ (R_{X+1}) and subsequently a fourth hydrogenolysis product stream (N^{th} hydrogenolysis product stream) is produced under conditions of butane hydrogenolysis.

The N^{th} hydrogenolysis product stream is the final hydrogenolysis product stream from which the hydrogenolysis products are recovered to obtain the one or more desired hydrogenolysis products. For example, in some embodiments of the invention, when the number of reactors in series is 4 (N=2), the second hydrogenolysis product stream is introduced in the third reactor R₃ to produce the third hydrogenolysis product stream.

The third hydrogenolysis product stream ((X)^{th} hydrogenolysis product stream) produced by the third reactor R₃ is introduced in the fourth reactor R₄ to produce the fourth or the final hydrogenolysis product stream. Accordingly, the fourth hydrogenolysis product stream is the N^{th} hydrogenolysis product stream and starting from the second hydrogenolysis product stream, the steps are repeated for a further two times till the fourth hydrogenolysis product stream is produced.

Accordingly, when N=2, the N^{th} hydrogenolysis product stream is the second hydrogenolysis product stream.

The final or the N^{th} hydrogenolysis product stream once produced in the last reactor R_{N}, at least a portion of the hydrogenolysis products from the N^{th} hydrogenolysis product stream is recovered to obtain the one or more desired hydrogenolysis products.

In some embodiments of the invention, the step of recovering at least a portion of the hydrogenolysis products from the N^{th} hydrogenolysis product stream, comprises:
a) feeding at least a portion of the N^{th} hydrogenolysis product stream to a separation unit and forming hydrogenolysis product stream, an unreacted hydrogen stream, a separated butane stream comprising i-butane and n-butane;
b) recovering at least a portion of the hydrogenolysis products from the hydrogenolysis product stream and obtaining one or more desired hydrogenolysis products;
c) feeding the separated butane stream into an isomerization unit and obtaining a n-butane rich stream; and
d) recirculating back at least a portion of the n-butane rich stream and the unreacted hydrogen stream to one or more reactors R₁ to R_{N}.

Preferably, the separated butane stream comprising i-butane and n-butane when introduced into the isomerization unit, the i-butane is converted to n-butane using known isomerization methodology, resulting in the formation of the n-butane rich stream.

In some embodiments of the invention, an additional butane feed and/or hydrogen feed is introduced into the (X+1) reactor (R_{X+1}), along with the (X)^{th} hydrogenolysis product stream. For example, when N=4, an additional butane feed and/or hydrogen feed may be introduced into any one R₂, R₃ or R₄ rectors. Preferably, in some embodiments of the invention, during the step of recovery of recovering at least a portion of the hydrogenolysis products from the N^{th} hydrogenolysis product stream, the n-butane rich stream and the unreacted hydrogen so produced may be recirculated back to the one or more reactors R₁ to R_{N}.

Alternatively, in some embodiments of the invention, the step of recovering at least a portion of the hydrogenolysis products from the N^{th} hydrogenolysis product stream, comprises the step of feeding at least a portion of the N^{th} hydrogenolysis product stream to a hydrocracking reactor and obtaining a hydrocracking product stream comprising one or more desired hydrogenolysis products.

In some embodiments of the invention, the one or more desired hydrogenolysis products are selected from ethane, propane, methane and mixtures thereof. Preferably in some embodiments of the invention, the one or more desired hydrogenolysis product is ethane produced at a selectivity of ≥60.0%, preferably ≥65.0%, preferably ≥75.0%, with regard to a total molar concentration of all hydrogenolysis products.

The overall n-butane conversion achieved by the process is sufficiently high for example, at least 60% of the n-butane present in the initial feed stream may be converted under conditions of hydrogenolysis. Accordingly, in some embodiments of the invention, the ratio of the molar concentration of n-butane present in the N^{th} hydrogenolysis product stream to the molar concentration of n-butane present in the initial feed stream is ≤0.4, preferably ≤0.3, preferably ≤0.2, preferably ≤0.1, preferably ≤0.05.

The inventors surprisingly found that when each of the ‘N’ reactors in series, except the first reactor, is purposefully configured to have a progressively increasing amount of catalyst loading compared to the immediately preceding reactor in the series, the process of butane hydrogenolysis of the present invention, results in high n-butane conversion while reducing the risks of thermal runaway. Accordingly, a reactor configured to receive a hydrogenolysis product stream denoted as R_{X+1} has ≥5% and ≤150%, preferably ≥15% and ≤120%, preferably ≥25% and ≤110%, preferably ≥30% and ≤80%, of higher catalyst loading than the catalyst loading of the immediately preceding reactor R_{X} configured to produce the hydrogenolysis product stream. In other words, in some embodiments of the of the invention, each reactor from R₂ to R_{N} has a catalyst loading (CL) that is ≥5% and ≤150%, preferably ≥15% and ≤120%, preferably ≥25% and ≤110%, preferably ≥30% and ≤80% higher than the catalyst loading of the immediately preceding reactor.

The increase in catalyst loading may for example be calculated by using the formula: ((CLsr−CLpr)/CLpr)×100, where CLsr is the catalyst loading of the subsequent reactor and CLpr is the catalyst loading of the preceding reactor, where CLsr>CLpr.

In some embodiments of the invention, the first reactor R₁ is operated under a condition of reactor temperature rise of ≤110° C., preferably ≤100° C., preferably ≤90° C., preferably ≤70° C., preferably ≤60° C., preferably ≤50° C., preferably ≤40° C., when an initial reactor inlet temperature (Tᵢₙₗₑₜ₁) of the first reactor R₁ is increased by an amount of ≥0.5° C. and ≤3.0° C., preferably ≥1.0° C. and ≤3.0° C., preferably ≥2.0° C. and ≤2.5° C., wherein the reactor temperature rise is determined as (Tₒᵤₜₗₑₜ₁−Tᵢₙₗₑₜ₁) where Tₒᵤₜₗₑₜ₁ is the outlet reactor temperature of the first reactor R₁ and Tᵢₙₗₑₜ₁ is the initial inlet temperature of the first reactor R₁.

Preferably, in some embodiments of the invention, the first reactor R₁ is operated under a condition of reactor temperature rise of ≥20° C. and ≤110° C., preferably ≥20° C. and ≤100° C., preferably ≥20° C. and ≤90° C., preferably ≥20° C. and ≤70° C., preferably ≤60° C., preferably ≥20° C. and ≤50° C., preferably ≥20° C. and ≤40° C., when an initial reactor inlet temperature (Tᵢₙₗₑₜ₁) of the first reactor R₁ is increased by an amount of ≥0.5° C. and ≤3.0° C., preferably ≥1.0° C. and ≤3.0° C., preferably ≥2.0° C. and ≤2.5° C., wherein the reactor temperature rise is determined as (Tₒᵤₜₗₑₜ₁−Tᵢₙₗₑₜ₁) where Tₒᵤₜₗₑₜ₁ is the outlet reactor temperature of the first reactor R₁ and Tᵢₙₗₑₜ₁ is the initial inlet temperature of the first reactor R₁.

The effect of progressively increasing the catalyst loading and reactor temperature rise across the reactors reduces the risks of thermal runaway in the first rector R₁, when the catalyst loading and the reactor temperature rise of the second reactor is suitably increased with regard to the catalyst loading and the reactor temperature rise of the first reactor. Mitigating the risks of thermal runaway in the first reactor R₁ may be particularly useful, as the first reactor has the highest risk for thermal runaway as compared to any of the other reactors in the series, owing to the higher concentration of the reactants (butane feed and hydrogen feed) introduced in the first reactor compared to the remaining reactors in the series.

The n-butane conversion in the first reactor R₁ may be sufficiently controlled for mitigating the risks of thermal runaway resulting in an n-butane conversion of at least 5.0%. Accordingly, the ratio of the molar concentration of n-butane present in a first hydrogenolysis product stream produced in the first reactor R₁ (first hydrogenolysis product stream) to the concentration of n-butane present in the initial feed stream is ≤0.95, preferably ≤0.85, preferably ≤0.70, preferably ≤0.5.

As an additional option to mitigate thermal runaway, the operating condition of butane feed in terms of the Weight Hourly Space Velocity (WHSV) may be adjusted to maximize n-butane conversion while mitigating any risks of thermal runaway. The Weight Hourly Space Velocity (WHSV) may be determined by dividing mass flow of butane expressed in (kg/hour) by catalyst load expressed in (kg).

In some embodiments of the invention, the reactor R_{X+1}, configured to receive a hydrogenolysis product stream from the immediate preceding reactor R_{X}, is operated at butane feed stream based weight hourly space velocity (WHSV) of ≥5% and ≤70%, preferably ≥10% and ≤60%, preferably ≥20% and ≤60%, lower than the weight hourly space velocity (WHSV) being operated at the reactor R_{X}. In other words, each reactor from R₂ to R_{N} is operated at a butane feed stream based weight hourly space velocity (WHSV) of ≥5% and ≤70%, preferably ≥10% and ≤60%, preferably ≥20% and ≤60%, lower than the butane feed stream based weight hourly space velocity (WHSV) being operated at the immediately preceding reactor.

The lowering of Weight Hourly Space Velocity may for example be calculated by using the formula: ((WHSVsr−WHSVpr)/WHSVpr)×100, where WHSVsr is the Weight Hourly Space Velocity of the subsequent reactor and WHSVpr is the Weight Hourly Space Velocity of the preceding reactor, where WHSVsr<WHSVpr.

Reactor conditions of the present invention can include temperature, pressure, a butane feed based WHSV, or combinations thereof. In some embodiments of the invention, when a series of ‘N’ reactors are used, the reactor(s) inlet(s) temperatures for each reactor can range from 240° C. to about 300° C., 245° C. to 290° C., 250° C. to 285° C., 260° C. to 280° C., or any value or range there between. In some preferred embodiments of the invention, the reactor inlet temperature of each reactor in the series of ‘N’ reactors is 245° C. to 290° C.

In some embodiments of the invention, pressures can range from about 101 kPa (1.01 Bar) to 1000 kPa (10 Bar), 200 kPa to 100 kPa, 500 kPa to 1000 kPa, or any range or value there between.

In some embodiments of the invention, butane feed-based WHSV for each of the ‘N’ reactors in the series can range from about 1 h⁻¹ to about 50 h⁻¹, 1 h⁻¹, 2 h⁻¹, 3 h⁻¹, 4 h⁻¹, 5 h⁻¹, 6 h⁻¹, 7 h⁻¹, 8 h⁻¹, 9 h⁻¹, 10 h⁻¹, 20 h⁻¹, 21 h⁻¹, 22 h⁻¹, 23 h⁻¹, 24 h⁻¹, 25 h⁻¹, 26 h⁻¹, 27 h⁻¹, 28 h⁻¹, 29 h⁻¹, 30 h⁻¹, 35 h⁻¹, 40 h⁻¹, 45 h⁻¹, 50 h⁻¹ or any range or value there between.

Accordingly, the butane feed-based WHSV for the remaining reactors in the series of ‘N’ reactors starting from the second reactor R₂ can be determined using the limitation that the reactor R_{X+1}, configured to receive a hydrogenolysis product stream from the immediate preceding reactor R_{X}, is operated at butane feed stream based weight hourly space velocity (WHSV) of ≥5% and ≤70%, preferably ≥10% and ≤60%, preferably ≥20% and ≤60%, lower than the weight hourly space velocity (WHSV) being operated at the reactor R_{X}.

Each of the ‘N’ reactors in series can include one or more heating and/or cooling devices (e.g., insulation, electrical heaters, jacketed heat exchangers in the wall) or controllers (e.g., computers, flow valves, automated values, etc.) that can be used to control the reaction temperature, the reactor inlet temperature and pressure of the reaction mixture.

Accordingly, in some embodiments of the invention, a reactor configured to receive a hydrogenolysis product stream is a reactor R_{X+1} having ≥1° C. and ≤50° C., preferably ≥2° C. and ≤40° C., preferably ≥5° C. and ≤35° C. of higher reactor temperature rise than the reactor temperature rise of the immediately preceding reactor R_{X} configured to produce the hydrogenolysis product stream. In other words, in some embodiments of the invention, the process is performed such that each reactor from R₂ to R_{N} has a reactor temperature rise of 1° C. and ≤50° C., preferably ≥2° C. and ≤40° C., preferably ≥5° C. and ≤35° C. higher than the reactor temperature rise of the immediately preceding reactor.

The calibrated increase in the reactor temperature rise can be achieved by controlling the temperature at the reactor inlet by any suitable method such as using heat exchangers, adjusting the catalyst load in the reactor and the Weight Hourly Space Velocity (WHSV) of the butane feed stream.

Without wishing to be bound by any specific theory, the purposeful control of catalyst loading in each of the reactors along with the calibrated increase in reactor temperature rise across the reactors R₁ to R_{N}, results in the mitigation of risks in thermal runaway while ensuring high butane conversion and hydrogenolysis product selectivity in particular of ethane.

In some preferred embodiments of the invention, the reactor system for conducting the process of hydrogenolysis comprises a plurality of 4 reactors (N=4) arranged in a series, wherein the first reactor of the series is denoted as R₁, the fourth reactor of the series is denoted as R₄, and the remaining reactors in the series are denoted as R₂ and R₃, wherein the second reactor R₂ has ≥25% and ≤110%, preferably ≥35% and ≤80%, preferably ≥40% and ≤70%, preferably ≥50% and ≤70%, of higher catalyst loading than the catalyst loading of the first reactor R₁, wherein the initial feed stream is introduced into the first reactor (R₁) and one or more desired hydrogenolysis products is obtained from a fourth hydrogenolysis product stream produced in the fourth reactor R₄.

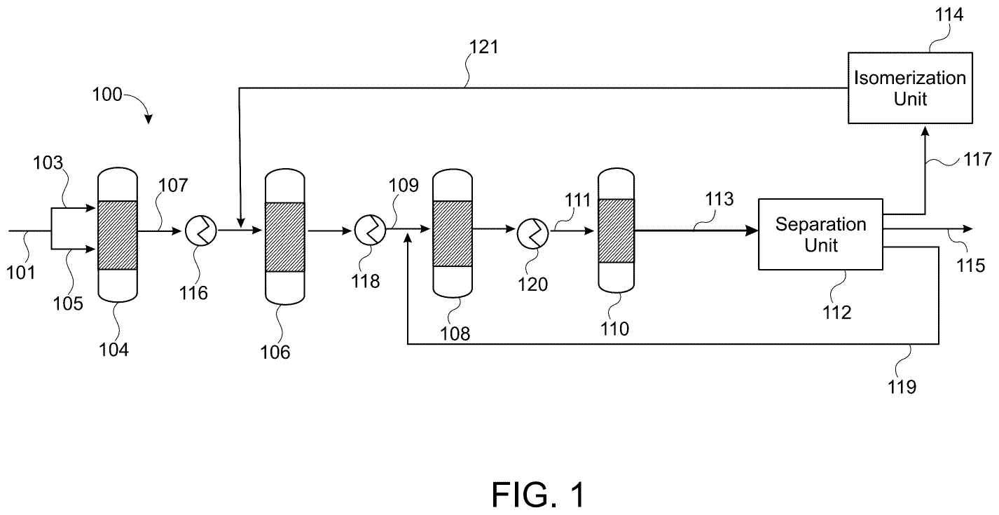

Referring to FIG. 1, in some preferred embodiments of the invention, the invention relates to a process for butane hydrogenolysis for producing one or more desired hydrogenolysis products using four fixed bed reactors arranged in series. The process steps may for example include the steps of:
a) providing a reactor system (100) comprising a plurality of 4 reactors (104), (106), (108) and (110) arranged in a series, wherein ‘N’ is 4; wherein the first reactor of the series is (104) denoted as R₁, the last reactor of the series is the fourth reactor (110) denoted as R₄, the penultimate reactor of the series is the third reactor (108) denoted as R₃ and the remaining reactor is the second reactor R₂ denoted as (106);
b) introducing the initial feed stream (101) comprising a butane feed (103) and hydrogen feed (105), into the first reactor (104) and producing the first hydrogenolysis product stream (107) under conditions butane hydrogenolysis;
c) the first hydrogenolysis product stream (107) may be introduced into the second reactor (106) to produce a second hydrogenolysis product stream (109) under conditions of butane hydrogenolysis;
d) the second hydrogenolysis product stream (109) may be introduced into the third reactor (108) to produce a third hydrogenolysis product stream (111) under conditions of butane hydrogenolysis; and
e) the third hydrogenolysis product stream (111) may be introduced into the fourth reactor (110) to produce a fourth hydrogenolysis product stream (113) under conditions of butane hydrogenolysis; and
f) recovering at least a portion of the hydrogenolysis products from the fourth hydrogenolysis product stream (113) and obtaining the one or more desired hydrogenolysis products, preferably the one or more desired hydrogenolysis products is ethane.

In some embodiments of the invention, the conditions of butane hydrogenolysis for the first reactor (104) includes an inlet temperature of ≥240° C. and ≤300° C., preferably 265° C. and ≤275° C. a butane feed stream based weight hourly space velocity (WHSV) of ≥15 h⁻¹ and ≤40 h⁻¹ preferably ≥15 h⁻¹ and ≤35 h⁻¹ and a pressure of 101 kPa (1.01 bar) to 1000 kPa (10 bar).

In some embodiments of the invention, the conditions of butane hydrogenolysis for the second reactor (106) includes an inlet temperature of ≥240° C. and ≤300° C., preferably ≥260° C. and ≤265° C. a butane feed stream based weight hourly space velocity (WHSV) of ≥10 h⁻¹ and ≤20 h⁻¹ and a pressure of 101 kPa (1.01 bar) to 1000 kPa (10 bar).

In some embodiments of the invention, the conditions of butane hydrogenolysis for the third reactor (108) includes an inlet temperature of ≥240° C. and ≤300° C., preferably ≥260° C. and ≤265° C., a butane feed stream based weight hourly space velocity (WHSV) of ≥5 h⁻¹ and ≤10 h⁻¹ and a pressure of 101 kPa (1.01 bar) to 1000 kPa (10 bar).

In some embodiments of the invention, the conditions of butane hydrogenolysis for the fourth reactor (110) includes an inlet temperature of ≥240° C. and ≤300° C., preferably ≥260° C. and ≤265° C., a butane feed stream based weight hourly space velocity (WHSV) of ≥1 h⁻¹ and ≤5 h⁻¹ and a pressure of 101 kPa (1.01 bar) to 1000 kPa (10 bar).

In some embodiments of the invention, the step of recovering the hydrogenolysis products from the fourth hydrogenolysis product stream (113) includes the step of:
a) feeding the fourth hydrogenolysis product stream (113) to a separation unit (112) and forming the hydrogenolysis product stream (115), the unreacted hydrogen stream (119), and the separated butane stream (117) comprising i-butane and n-butane;
b) recovering at least a portion of the hydrogenolysis products from the hydrogenolysis product stream (115) and obtaining one or more desired hydrogenolysis products, preferably the one or more desired hydrogenolysis products is ethane;
c) feeding the separated butane stream (117) into an isomerization unit (114) and obtaining a n-butane rich stream (121); and
d) recirculating back the n-butane rich stream (121) to the second reactor (106) and the unreacted hydrogen stream (119) to the third reactor (108).

In some embodiments of the invention, the n-butane rich stream (121) may be recirculated back to the first reactor (104) (not shown in FIG. 1). In some embodiments of the invention, the n-butane rich stream (121) may be recirculated back to the second reactor (106) (not shown in FIG. 1). In some embodiments of the invention, the n-butane rich stream (121) may be recirculated back to the third reactor (108) (not shown in FIG. 1).

In some embodiments of the invention, the unreacted hydrogen stream (119) may be recirculated back to the first reactor (104) (not shown in FIG. 1). In some embodiments of the invention, the unreacted hydrogen stream (119) may be recirculated back to the second reactor (106) (not shown in FIG. 1).

In some embodiments of the invention, the second reactor R₂ (106) has ≥5% and ≤150%, preferably ≥15% and ≤120%, preferably ≥25% and ≤110% preferably ≥30% and ≤80%, of higher catalyst loading than the catalyst loading of the first reactor R₁ (104). In some embodiments of the invention, the third reactor R₃ (108) has ≥5% and ≤150%, preferably ≥15% and ≤120%, preferably ≥25% and ≤110% preferably ≥30% and ≤80%, of higher catalyst loading than the catalyst loading of the second reactor R₂ (106). In some embodiments of the invention, the fourth reactor R₄ (110) has ≥5% and ≤150%, preferably ≥15% and ≤120%, preferably ≥25% and ≤110% preferably ≥30% and ≤80%, of higher catalyst loading than the catalyst loading of the third reactor R₃ (108).

Preferably, in some embodiments of the invention, the second reactor (106) R₂ has ≥25% and ≤110%, preferably ≥35% and ≤80%, preferably ≥40% and ≤70%, preferably ≥50% and ≤70%, of higher catalyst loading than the catalyst loading of the first reactor R₁ (104). Preferably, in some embodiments of the invention, the second reactor (106) R₂ has preferably ≥40% and ≤70%, preferably ≥50% and ≤70%, of higher catalyst loading than the catalyst loading of the first reactor R₁ (104).

In some embodiments of the invention, the second reactor (106) R₂ has ≥1° C. and ≤50° C., preferably ≥2° C. and ≤40° C., preferably ≥5° C. and ≤35° C. of higher reactor temperature rise than the reactor temperature rise of the first reactor (104) R₁. In some embodiments of the invention, the third reactor (108) R₃ has ≥1° C. and ≤50° C., preferably ≥2° C. and ≤40° C., preferably ≥5° C. and ≤35° C. of higher reactor temperature rise than the reactor temperature rise of the second reactor (106) R₂.

In some embodiments of the invention, the fourth reactor (110) R₄ has ≥1° C. and ≤50° C., preferably ≥2° C. and ≤40° C., preferably ≥5° C. and ≤35° C. of higher reactor temperature rise than the reactor temperature rise of the third reactor (108) R₃.

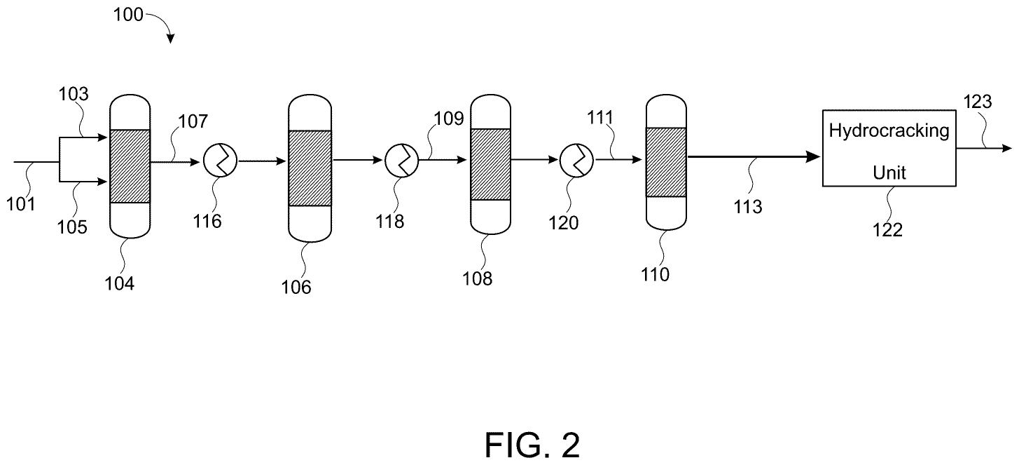

Referring to FIG. 2, in some embodiments of the invention, the step of recovering the hydrogenolysis products from the fourth hydrogenolysis product stream (113) includes the step of feeding at least a portion of the fourth hydrogenolysis product stream (113) to a hydrocracking reactor (122) and obtaining a hydrocracking product stream (123) comprising one or more desired hydrogenolysis products, preferably the one or more desired hydrogenolysis products is ethane.

In some embodiments of the invention, the first hydrogenolysis product stream (107) is passed through a heat exchanger (116) prior to introducing the first hydrogenolysis product stream (107) into the second reactor (106). In some embodiments of the invention, the second hydrogenolysis product stream (109) is passed through a heat exchanger (118) prior to introducing the second hydrogenolysis product stream (109) into the third reactor (108). In some embodiments of the invention, the third hydrogenolysis product stream (111) is passed through a heat exchanger (120) prior to introducing the third hydrogenolysis product stream (111) into the fourth reactor (110). Although FIG. 1 and FIG. 2 show the presence of heat exchangers, in some embodiments of the invention, in some other embodiments of the invention, heat exchangers may not be required for accomplishing the objectives of the present invention.

In some preferred embodiments of the invention, the invention relates to a process for butane hydrogenolysis for producing one or more desired hydrogenolysis products using five fixed bed reactors arranged in series. The process steps may for example include the steps of:
a) providing a reactor system comprising a plurality of 5 reactors arranged in a series, wherein ‘N’ is 5; wherein the first reactor of the series is denoted as R₁, the last reactor of the series is the fifth reactor denoted as R₅, the penultimate reactor of the series is the fourth reactor denoted as R₄ and the remaining reactors are the second reactor R₂ and the third reactor R₃;
b) introducing the initial feed stream comprising a butane feed and hydrogen feed into the first reactor and producing the first hydrogenolysis product stream under conditions butane hydrogenolysis;
c) the first hydrogenolysis product stream may be introduced into the second reactor to produce a second hydrogenolysis product stream under conditions of butane hydrogenolysis;
d) the second hydrogenolysis product stream may be introduced into the third reactor to produce a third hydrogenolysis product stream under conditions of butane hydrogenolysis;
e) the third hydrogenolysis product stream may be introduced into the fourth reactor to produce a fourth hydrogenolysis product stream under conditions of butane hydrogenolysis;
f) the fourth hydrogenolysis product stream may be introduced into the fifth reactor to produce a fifth hydrogenolysis product stream under conditions of butane hydrogenolysis;
g) recovering at least a portion of the hydrogenolysis products from the fifth hydrogenolysis product stream and obtaining the one or more desired hydrogenolysis products, preferably the one or more desired hydrogenolysis products is ethane. The one or more desired hydrogenolysis products are selected from ethane, propane, methane and mixtures thereof

Preferably, in some embodiments of the invention, for the reactor system having five reactors, the second reactor R₂ has ≥25% and ≤110%, preferably ≥35% and ≤80%, preferably ≥40% and ≤70%, preferably ≥50% and ≤70%, of higher catalyst loading than the catalyst loading of the first reactor R₁. Preferably, in some embodiments of the invention, the second reactor R₂ has preferably ≥40% and ≤70%, preferably ≥50% and ≤70%, of higher catalyst loading than the catalyst loading of the first reactor R₁. In some embodiments of the invention, for the reactor system having five reactors, a reactor configured to receive a hydrogenolysis product stream has a ≥1° C. and ≤50° C., preferably ≥2° C. and ≤40° C., preferably ≥5° C. and ≤35° C. of higher reactor temperature rise than the reactor temperature rise of the immediately preceding reactor configured to produce the hydrogenolysis product stream.

In some preferred embodiments of the invention, the invention relates to a process for butane hydrogenolysis for producing one or more desired hydrogenolysis products using two fixed bed reactors arranged in series. The process steps may for example include the steps of:
a) providing a reactor system comprising a plurality of 2 reactors arranged in a series, wherein ‘N’ is 2; wherein the first reactor of the series is denoted as R₁, the last reactor of the series is the second reactor denoted as R₂;
b) introducing the initial feed stream comprising a butane feed and hydrogen feed into the first reactor and producing the first hydrogenolysis product stream under conditions butane hydrogenolysis;
c) the first hydrogenolysis product stream may be introduced into the second reactor to produce a second hydrogenolysis product stream under conditions of butane hydrogenolysis;
d) recovering at least a portion of the hydrogenolysis products from the second hydrogenolysis product stream and obtaining the one or more desired hydrogenolysis products, preferably the one or more desired hydrogenolysis products is ethane.

Preferably, in some embodiments of the invention, for the reactor system having 2 reactors, the second reactor R₂ has ≥25% and ≤110%, preferably ≥35% and ≤80%, preferably ≥40% and ≤70%, preferably ≥50% and ≤70%, of higher catalyst loading than the catalyst loading of the first reactor R₁. Preferably, in some embodiments of the invention, the second reactor R₂ has preferably ≥40% and ≤70%, preferably ≥50% and ≤70%, of higher catalyst loading than the catalyst loading of the first reactor R₁.

In some embodiments of the invention, the second reactor R₂ configured to receive a hydrogenolysis product stream has a ≥1° C. and ≤50° C., preferably ≥2° C. and ≤40° C., preferably ≥5° C. and ≤35° C. of higher reactor temperature rise than the reactor temperature rise of the immediately preceding first reactor R₁ configured to produce the hydrogenolysis product stream.

In some embodiments of the invention, the methane diluent is added to at least one of the reactors 104, 106, 108, 110. The amount of methane required to reduce the temperature rise during butane conversion is suitably adjusted. In some embodiments of the invention, the methane diluent is part of at least one of a) the initial feed stream 101 b) at least one of the hydrogenolysis product streams 107, 109, 111 c) introduced in to at least one of the reactors 104, 106, 108, 110. The amount of methane required to reduce the temperature rise during butane conversion is suitably adjusted. Although other diluents may be used, including mixed gas diluents, methane is preferred Methane is preferred because methane is already generated in the reaction, the separation for methane is not difficult and should already be in place in the process, and methane has relatively high heat capacity. Other possible diluents include nitrogen and other hydrocarbons.

Specific examples demonstrating some of the embodiments of the invention are included below. The examples are for illustrative purposes only and are not intended to limit the invention. It should be understood that the embodiments and the aspects disclosed herein are not mutually exclusive and such aspects and embodiments can be combined in any way. Those of ordinary skill in the art will readily recognize parameters that can be changed or modified to yield essentially the same results.

### Example 1

Purpose: To demonstrate a process of butane hydrogenolysis in accordance with the features of the present invention.

For the purpose of the example, four case simulations were made using the software Python based Pyomo, where ‘IE’ represents a process simulated in accordance with the process of present invention, where catalyst loadings and reactor temperature rise were configured to increase progressively in accordance with the invention. CE1-CE3 represent comparative examples. For the purpose of the examples, as the simulations assume identical reactors in series, the catalyst loading is simply the catalyst mass in a given reactor.

For each of the four cases (IE, CE1, CE2 and CE3) involved a butane hydrogenolysis process having four fixed bed reactors arranged in series. For the purpose of the present invention, for each of the processes IE, CE1, CE2, and CE3 four dimensionally identical fixed bed reactors were used each having identical volume.

The catalyst used for each of the process was a catalyst composition having 0.3 wt. % Pt-0.3 wt. % Ir/γ-Al₂O₃-HZSM-5. The catalyst carrier was composed of 80 wt. % H-ZSM-5 and 20 wt. % T-Al₂O₃. The H-ZSM-5 had a SiO₂/Al₂O₃ of 280.

Process steps: Referring to FIG. 1 or FIG. 2, the process steps for the butane hydrogenolysis for each of case reference (IE, CE1-CE3) included the steps of:
a) a reactor system (100) was provided comprising 4 reactors (104), (106), (108) and (110) arranged in a series; wherein the first reactor of the series was (104) denoted as R₁, the last reactor of the series was the fourth reactor (110) denoted as R₄, the penultimate reactor of the series was the third reactor (108) denoted as R₃ and the remaining reactor was the second reactor R₂ denoted as (106);
b) an initial feed stream (101) comprising a butane feed (103) and a hydrogen feed (105), was introduced into a first reactor (104) and a first hydrogenolysis product stream (107) was produced under conditions butane hydrogenolysis;
c) the first hydrogenolysis product stream (107) was introduced into a second reactor (106) and a second hydrogenolysis product stream (109) was produced under conditions of butane hydrogenolysis;
d) the second hydrogenolysis product stream (109) was introduced into a third reactor (108) to produce a third hydrogenolysis product stream (111) under conditions of butane hydrogenolysis; and
e) the third hydrogenolysis product stream (111) was introduced into a fourth reactor (110) to produce a fourth hydrogenolysis product stream (113) under conditions of butane hydrogenolysis; and
f) recovering a hydrogenolysis products from the fourth hydrogenolysis product stream (113) and obtaining the one or more desired hydrogenolysis products, preferably ethane.

Operational parameters: For each of the cases, pressure was kept constant at 7.9 bar for all the reactors. The initial feed stream had a ratio of n-butane to i-butane of 6:4. The total WHSV across all the four reactors was maintained at 3.0 h⁻¹. The operating parameters for the remaining cases are provided in the table below.

TABLE 1

| | | Compar- | Compar- | Compar- | |
|---|---|---|---|---|---|
| | | ative | ative | ative | Inventive |
| Parameters | Reactors | CE1 | CE2 | CE3 | IE |
| Catalyst | R1 | 0.775 | 0.775 | 0.488 | 0.317 |
| Load [kg] | R2 | 0.775 | 0.775 | 0.575 | 0.531 |
| | R3 | 0.775 | 0.775 | 0.757 | 0.751 |
| | R4 | 0.775 | 0.775 | 1.280 | 1.500 |
| Inlet | R1 | 257.60 | 256.21 | 265.02 | 270.54 |
| Temperature | R2 | 260.24 | 258.62 | 265.02 | 264.66 |
| [° C.] | R3 | 264.70 | 262.64 | 265.02 | 263.09 |
| | R4 | 273.00 | 271.10 | 265.02 | 260.55 |
| Butane feed | R1 | 12.0 | 12.0 | 19.0 | 29.2 |
| stream | R2 | 9.8 | 10.4 | 10.5 | 12.5 |
| (WHSV) | R3 | 7.7 | 8.5 | 7.9 | 8.8 |
| h⁻¹ | R4 | 5.8 | 6.3 | 3.5 | 3.2 |
| Reactor | R1 | 50 | 35 | 50 | 35 |
| Temperature | R2 | 50 | 45 | 50 | 45 |
| Rise | R3 | 50 | 55 | 50 | 55 |
| ΔT [° C.] | R4 | 50 | 65 | 50 | 65 |

The table below provides the percentage increase or decrease of a parameter relative to the preceding reactor. For reactor R1, the value for a parameter has been provided as given in the above table.

TABLE 2

| | | Compar- | Compar- | Compar- | |
|---|---|---|---|---|---|
| Param- | Reac- | ative | ative | ative | Inventive |
| eters | tors | CE1 | CE2 | CE3 | IE |
| Catalyst | R1 | 0.775 | 0.775 | 0.488 | 0.317 |
| Load [kg] | R2 | 0% | 0% | 18% higher | 68% higher |
| | | | | than R1 | than R1 |
| | R3 | 0% | 0% | 32% higher | 41% higher |
| | | | | than R2 | than R2 |
| | R4 | 0% | 0% | 69% higher | 100% higher |
| | | | | than R3 | than R3 |
| Butane | R1 | 12 | 12 | 19 | 29.2 |
| feed | R2 | 18% lower | 13% lower | 45% lower | 57% lower |
| stream | | than R1 | than R1 | than R1 | than R1 |
| (WHSV) | R3 | 21% lower | 18% lower | 25% lower | 30% lower |
| h⁻¹ | | than R1 | than R2 | than R2 | than R2 |
| | R4 | 25% lower | 26% lower | 56% lower | 64% lower |
| | | than R3 | than R3 | than R3 | than R3 |
| Reactor | R1 | 50 | 35 | 50 | 35 |
| Temper- | R2 | 0% | 29% higher | 0% | 29% higher |
| ature | | | than R1 | | than R1 |
| Rise | R3 | 0% | 22% higher | 0% | 22% higher |
| [° C.] | | | than R2 | | than R2 |
| | R4 | 0% | 18% higher | 0% | 18% higher |
| | | | than R3 | | than R3 |

Results: The results in terms of the thermal runaway, n-butane conversion and ethane selectivity is shown below:

TABLE 3

| | | Comparative | Comparative | Comparative | Inventive |
|---|---|---|---|---|---|
| | | CE1 | CE2 | CE3 | IE |
| n-Butane conversion | R1 | 91% | 40% | 91% | 33% |
| at +2.0° C. increase of the | | | | | |
| reactor inlet temperature | | | | | |
| of the reactor R1 | | | | | |
| Ethane selectivity at +2.0° | R1 | 61% | 73% | 55% | 72% |
| C. increase of the reactor | | | | | |
| inlet temperature for the | | | | | |
| reactor R1 | | | | | |

| | | | | |
|---|---|---|---|---|
| Reactor temperature rise when there | 87 | 44 | 79 | 43 |
| is a +1.0° C. increase of the reactor | | | | |
| inlet temperature for the reactor R1 | | | | |
| Reactor temperature rise when there | Thermal | 64 | Thermal | 56 |
| is a +2.0° C. increase of the reactor | runaway | | runaway | |
| inlet temperature for the reactor R1 | | | | |
| Reactor temperature rise when there | Thermal | Thermal | Thermal | 70 |
| is a +2.5° C. increase of the reactor | runaway | runaway | runaway | |
| inlet temperature for the reactor R1 | | | | |

The inventive process IE, is distinguished from the remaining comparative process, where the inventive process has a progressive increase in both catalyst load and a progressive increase of reactor temperature rise across the reactors along the flow of the reactants and product streams. From the above table it is evident that for the inventive process (IE), the process parameters are configured in such a manner than even with a 2.5° C. change in temperature at the inlet of the first reactor, thermal runaway does not occur, thereby ensuring the desired stability of the process and lower sensitivity to fluctuation of the reactor inlet temperature.

Further, for the inventive process, IE, the selectivity to ethane at the first reactor is desirably high at almost 72%. The selectivity of ethane at the first reactor is particularly important as butane concentration is highest in the first reactor R1 and a suitable selectivity of ethane at the first reactor R1, would help impart the desired selectivity to the overall selectivity of ethane for a particular hydrogenolysis process. The inventive process IE, has a similar selectivity at the first reactor as that of the process of CE2. However, it is seen that the process of CE2, is more sensitive to fluctuation of inlet reactor temperature, where even a 2.5° C. increase in reactor temperature leads to thermal runaway. Therefore, from the examples, it is clearly evidenced that with progressive increase in catalyst loading and reactor temperature rise within the limits prescribed in the invention, would result in meeting the one or more objectives of the present invention.

### Example 2

To demonstrate the benefit of methane introduction as a dilution into the hydrogenolysis reactors, the following example is provided. It should be understood that the data are all from simulations. The methane acts as a heat carrier, which reduces the temperature rise over the same butane conversion. As a result, with the same temperature rise limit, the dilution can increase the butane conversion per reactor safely. This can also reduce the number of reactors required to achieve the same overall butane conversion.

Below two tables (Table 4, Case1 and Table 5, (Case2) show simulation results. All simulations used the same weight hourly space velocity (WHSV=3) and number of reactors (R1, R2, R3, and R4). In the tables, C1, ΔT, Ti, X(nC4), and S(C2) indicate CH4/C4 molar ratio (C4=i-butane+n-butane) at the first reactor, temperature rise per reactor, inlet temperature, n-butane conversion, and ethane selectivity, respectively. In these simulations, methane is only added at the first reactor. This is the preferred embodiment because since the first stage has highest reactant concentration and is most susceptible to runaway. Although the benefits are not expected to be as great, the diluent can be added to any one or more of the reactors. Case1 and Case2 correspond to total temperature rise of 220° C. and 240° C. in all four reactors. In the tables, C1(0%) is a reference without any methane diluent. Note that we introduced progressive temperature rise per reactor (e.g. 35, 50, 65, 70) as it can potentially reduce the risk of thermal runaway. In addition, interstage hydrogen injection was also introduced as it can mitigate the catalyst deactivation as previously disclosed.

In conclusion, the butane conversion per reactor increased with the methane dilution. In cases of the methane dilution more than 80% w.r.t butane concentration, we were able to achieve the similar (or target) butane conversion level (≥90%) with three reactors compared to the reference cases in Tables 4 and 5.

TABLE 4
Case1 - Total temperature rise = 220° C.

| | | C1(0%) | | | C1(50%) | | | C1(80%) | | | C1(100%) | | | C1(150%) | | |
|---|---|---|---|---|---|---|---|---|---|---|---|---|---|---|---|---|
| | ΔT | Ti[C] | X(nC4) | S(S2) | Ti[C] | X(nC4) | S(S2) | Ti[C] | X(nC4) | S(S2) | Ti[C] | X(nC4) | S(S2) | Ti[C] | X(nC4) | S(S2) |
| R1 | 35 | 256.2 | 21.0% | 73.5% | 259.3 | 24% | 73.4% | 261.03 | 25% | 73.3% | 262.3 | 26% | 73.2% | 265.3 | 29.1% | 73.1% |
| R2 | 50 | 259.0 | 50.0% | 73.9% | 262.2 | 56% | 73.9% | 263.98 | 59% | 73.9% | 265.4 | 62% | 73.9% | 268.9 | 67.1% | 73.9% |
| R3 | 65 | 263.6 | 82.2% | 75.1% | 267.7 | 89% | 75.6% | 270.30 | 92% | 75.8% | 272.3 | 93% | 76.0% | 277.7 | 96.0% | 76.3% |
| R4 | 70 | 274.0 | 97.7% | 77.2% | 280.6 | 98% | 76.8% | 284.22 | 98% | 76.0% | 286.8 | 99% | 75.4% | 292.7 | 98.7% | 73.6% |

TABLE 5
Case2 - Total temperature rise = 240° C.

| | | C1(0%) | | | C1(50%) | | | C1(80%) | | | C1(100%) | | | C1(150%) | | |
|---|---|---|---|---|---|---|---|---|---|---|---|---|---|---|---|---|
| | ΔT | Ti[C] | X(nC4) | S(S2) | Ti[C] | X(nC4) | S(S2) | Ti[C] | X(nC4) | S(S2) | Ti[C] | X(nC4) | S(S2) | Ti[C] | X(nC4) | S(S2) |
| R1 | 35 | 256.2 | 21% | 73.5% | 259.3 | 24% | 73.4% | 261.0 | 25% | 73.3% | 262.3 | 26% | 73.2% | 265.3 | 29.1% | 73.1% |
| R2 | 55 | 259.2 | 53% | 73.8% | 262.4 | 59% | 73.9% | 264.3 | 62% | 73.9% | 265.7 | 65% | 73.9% | 269.2 | 70.3% | 74.0% |
| R3 | 70 | 264.3 | 86% | 75.3% | 268.8 | 92% | 75.8% | 271.5 | 94% | 76.0% | 273.7 | 95% | 76.1% | 279.5 | 97.1% | 76.2% |
| R4 | 80 | 276.1 | 98% | 76.7% | 283.0 | 98% | 75.5% | 286.4 | 99% | 74.2% | 288.8 | 99% | 73.3% | 294.2 | 98.8% | 71.1% |

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A process for butane hydrogenolysis for producing one or more desired hydrogenolysis products, comprising:
a) providing a reactor system comprising a plurality of ‘N’ reactors arranged in a series, wherein ‘N’ is an integer ≤10; wherein the first reactor of the series is denoted as R₁, the last reactor of the series is denoted as R_{N}, the penultimate reactor of the series is denoted as R_{N-1} and the remaining reactors are denoted serially from 2 to (N-2);
b) introducing a feed stream comprising a butane feed and hydrogen feed, in a ‘X’ reactor denoted as R_{X}, and producing a (X)^{th} hydrogenolysis product stream under conditions of butane hydrogenolysis; wherein the (X)^{th} hydrogenolysis product stream comprises an unreacted butane feed, hydrogenolysis products, and unreacted hydrogen;
i. wherein R_{X} is any one of the reactors arranged in the series from reactor R₁ to reactor R_{N-1};
ii. wherein ‘X’ is an integer ranging from ≥1 and ≤N-1, wherein when ‘X’=1, the feed stream introduced in the first reactor R₁ is an initial feed stream being introduced into the reactor system;
c) introducing at least a portion of the (X)^{th} hydrogenolysis product stream to an immediate subsequent reactor, a (X+1) reactor denoted as R_{X+1}, and producing a (X+1)^{th} hydrogenolysis product stream under conditions of butane hydrogenolysis; wherein the (X+1)^{th} hydrogenolysis product stream comprises an unreacted butane feed, hydrogenolysis products, and unreacted hydrogen;
i. wherein the reactor R_{X+1} is an immediately subsequent reactor to the reactor R_{X};
d) provided if N is ≥2, repeating the process steps of b) and c) for a further N-2 times until a N^{th} hydrogenolysis product stream is produced in the R_{N} reactor; wherein the N^{th} hydrogenolysis product stream comprises unreacted butane, hydrogenolysis products, and unreacted hydrogen, provided that a feed stream introduced in the reactor R_{X+1} comprises at least a portion of the (X)^{th} hydrogenolysis product stream that is produced in the immediately preceding reactor R_{X},
i. provided if ‘N’ is 2, a second hydrogenolysis product stream produced by a second reactor R₂ is the N^{th} hydrogenolysis product stream;
e) recovering at least a portion of the hydrogenolysis products from the N^{th} hydrogenolysis product stream and obtaining one or more desired hydrogenolysis products;
wherein each of the reactors in series from R₁ to R_{N} comprises:
i. a reactor vessel; and
ii. a catalyst bed disposed in the reactor vessel, wherein the catalyst bed comprises: a butane hydrogenolysis catalyst, wherein the butane hydrogenolysis catalyst is loaded at a catalyst loading (CL); wherein each reactor from R₂ to R_{N} has a catalyst loading (CL) that is ≥5% and ≤150% higher than the catalyst loading of the immediately preceding reactor;
iii. wherein the process is performed such that each reactor from R₂ to R_{N} has a reactor temperature rise of ≥1° C. and ≤50° C. higher than the reactor temperature rise of the immediately preceding reactor; wherein the one or more desired hydrogenolysis products are selected from ethane, propane, methane and mixtures thereof

2. The process of claim 1, wherein each reactor from R₂ to R_{N} is operated at a butane feed stream based weight hourly space velocity (WHSV) of ≥5% and ≤70% lower than the butane feed stream based weight hourly space velocity (WHSV) being operated at the immediately preceding reactor; and/or the process further comprises introducing a diluent into one or more of the R₁ to R_{N} reactors.

3. The process of claim 1, wherein when X=1, the second reactor R₂ has ≥25% and ≤110% of higher catalyst loading than the catalyst loading of the first reactor R₁.

4. The process of claim 1, wherein the one or more desired hydrogenolysis product is ethane produced at a selectivity of ≥60.0% with regard to a total molar concentration of all hydrogenolysis products.

5. The process of claim 1, wherein an additional butane feed and/or hydrogen feed is introduced into the (X+1) reactor (R_{X+1}), along with the (X)^{th} hydrogenolysis product stream.

6. The process of claim 1, wherein the butane feed and/or the unreacted butane feed comprises a mixture of n-butane and/or i-butane.

7. The process of claim 1, wherein the ratio of the molar concentration of n-butane present in the N^{th} hydrogenolysis product stream to the molar concentration of n-butane present in the initial feed stream is ≤0.4.

8. The process of claim 1, wherein the ratio of the molar concentration of n-butane present in a first hydrogenolysis product stream produced in the first reactor R₁ (first hydrogenolysis product stream) to the molar concentration of n-butane present in the initial feed stream is ≤0.95.

9. The process of claim 1, wherein the first reactor R₁ is operated under a condition of reactor temperature rise of ≤110° C. when an initial reactor inlet temperature (Tᵢₙₗₑₜ₁) of the first reactor R₁ is increased by an amount of ≥0.5° C. and ≤3.0° C., wherein the reactor temperature rise is determined as (Tₒᵤₜₗₑₜ₁−Tᵢₙₗₑₜ₁) where Tₒᵤₜₗₑₜ₁ is the outlet reactor temperature of the first reactor R₁ and Tᵢₙₗₑₜ₁ is the initial inlet temperature of the first reactor R₁.

10. The process of claim 1, wherein the step of recovering at least a portion of the hydrogenolysis products from the N^{th} hydrogenolysis product stream, comprises:
a) feeding at least a portion of the N^{th} hydrogenolysis product stream to a separation unit and forming a hydrogenolysis product stream, an unreacted hydrogen stream, a separated butane stream comprising i-butane and n-butane;
b) recovering at least a portion of the hydrogenolysis products from the hydrogenolysis product stream and obtaining one or more desired hydrogenolysis products;
c) feeding the separated butane stream into an isomerization unit and obtaining a n-butane rich stream; and
d) recirculating back at least a portion of the n-butane rich stream and the unreacted hydrogen stream to one or more reactors R₁ to R_{N}.

11. The process of claim 1, wherein the step of recovering at least a portion of the hydrogenolysis products from the N^{th} hydrogenolysis product stream, comprises the step of feeding at least a portion of the N^{th} hydrogenolysis product stream to a hydrocracking reactor and obtaining a hydrocracking product stream comprising one or more desired hydrogenolysis products.

12. The process of claim 1, wherein the butane hydrogenolysis catalyst is selected from:
(i) a bimetallic supported catalyst comprising a support, a first catalytic metal, a second catalytic metal, and optionally a binder, wherein the first catalytic metal and the second catalytic metal are different,
(ii) a monometallic supported catalyst, the monometallic catalyst comprising a third catalytic metal, a support, and optionally binder, or
(iii) mixtures of (i) and (ii),
wherein the first catalytic metal, the second catalytic metal, and the third catalytic metal are each independently selected from iridium (Ir), platinum (Pt), rhodium (Rh), ruthenium (Ru), rhenium (Re), palladium (Pd), molybdenum (Mo), tungsten (W), nickel (Ni), or cobalt (Co), or any combination thereof.

13. The process of claim 12, wherein the butane hydrogenolysis catalyst comprises the bimetallic supported catalyst comprising Ir and Pt and wherein the support comprises alumina, a zeolite, or both, wherein the zeolite comprises ZSM-5, ZSM-11, Y, high-silica Y, USY, EU-1, EU-2, beta, L, ferrierite, CHA, SSZ-16, Nu-3, sigma-1, or silicalite-1, or any combination thereof, and optionally wherein the binder if present, is selected from alumina, titania, silica, or combinations thereof.

14. The process of claim 1, wherein the reactor system comprises a plurality of 4 reactors (N=4) arranged in a series, wherein the first reactor of the series is denoted as R₁, the fourth reactor of the series is denoted as R₄, and the remaining reactors in the series are denoted as R₂ and R₃, wherein the second reactor R₂ has ≥25% and ≤110% of higher catalyst loading than the catalyst loading of the first reactor R₁, wherein the initial feed stream is introduced into the first reactor (R₁) and one or more desired hydrogenolysis products is obtained from a fourth hydrogenolysis product stream produced in the fourth reactor R₄.

15. A reactor system for conducting the process of butane hydrogenolysis as claimed in claim 1, wherein the reactor system comprises a plurality of ‘N’ reactors arranged in a series, wherein ‘N’ is an integer ≤10; wherein the first reactor of the series is denoted as R₁, the last reactor of the series is denoted as R_{N}, the penultimate reactor of the series is denoted as R_{N-1} and the remaining reactors are denoted serially from 2 to (N-2);
a) wherein each of the reactors ranging from R₁ to R_{N} comprises:
i. a reactor vessel; and
ii. a catalyst bed disposed in the reactor vessel and extending along a flow direction of reactants, wherein the catalyst bed comprises a butane hydrogenolysis catalyst loaded at a catalyst load (CL); wherein each reactor from R₂ to R_{N} has a catalyst loading (CL) that is ≥5% and ≤150% higher than the catalyst loading of the immediately preceding reactor;
b) the first reactor R₁ is configured to operate under a condition of reactor temperature rise of ≤110° C. when an initial reactor inlet temperature (Tᵢₙₗₑₜ₁) of the first reactor R₁ is increased by an amount of ≥0.5° C. and ≤3.0° C., wherein the reactor temperature rise is determined as (Tₒᵤₜₗₑₜ₁−Tᵢₙₗₑₜ₁) where Tₒᵤₜₗₑₜ₁ is the outlet reactor temperature of the first reactor R₁ and Tᵢₙₗₑₜ₁ is the initial inlet temperature of the first reactor R₁; wherein the first reactor R₁ is configured to receive the initial feed stream;
c) wherein the reactor system is configured to achieve an overall n-butane conversion of ≥60.00%; and
d) wherein each reactor from R₂ to R_{N} is configured to have a reactor temperature rise of ≥1° C. and ≤50° C., higher than the reactor temperature rise of the immediately preceding reactor; and optionally wherein at least of one of the reactors R₁ to R_{N} has an inlet to receive a diluent gas.
